(19) United States
(12) Patent Application Publication
(10) Pub. No.: US 2026/0355463 A1
(43) Pub. Date: Nov. 12, 2026

(54) ROLLING MEMBRANE CATHETER WITH AT LEAST PARTLY AUTOMATED ROLLING MEMBRANE PRESSURE AND GUIDEWIRE RETRACTION

(71) Applicant: BIOTRONIK AG, Buelach (CH)

(72) Inventors: Matthias Wesselmann, Ruedlingen (CH); Claus Harder, Dormitz (DE); Azedeh Mehrabi, Meilen (CH)

(21) Appl. No.: 19/122,967

(22) PCT Filed: Nov. 23, 2023
(86) PCT No.: PCT/EP2023/082850

§ 371 Date: Apr. 21, 2025

(30) Foreign Application Priority Data
Dec. 12, 2022 (EP) 22212726.8

(51) Int. Cl. A61M 25/01; A61B 17/22

(52) U.S. Cl. CPC A61M 25/0119; A61B 17/22; A61B 2017/22048

(57) ABSTRACT

A rolling membrane catheter includes a first rolling membrane. A motion unit is configured to determine a motion state of the first rolling membrane. The rolling membrane catheter can couple with a fluid unit for determining a fluid pressure and/or a fluid flow of the first rolling membrane. The rolling membrane catheter can be adapted to automatically couple the fluid unit and the motion unit for synchronized operation at least in part based on the motion state, the fluid pressure, and/or the fluid flow.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/082850, which was filed Nov. 23, 2023, which application claimed priority from EP Application 2221276.8, which was filed Dec. 12, 2022.

### FIELD OF THE INVENTION

A field of the invention concerns catheter devices and devices to treat stenosis.

### BACKGROUND

Medical treatment of a stenosis via percutaneous coronary intervention (PCI) can involve the insertion of a catheter into a blood vessel at the position of the stenosis. Rolling membrane catheters allow catheter insertion into narrow cavities by everting a rolling membrane during a roll-out process. Typically, in a first step, a guidewire is introduced into the blood vessel and guided to the treatment site, e.g., the location of a stenosis. The rolling membrane of the rolling membrane catheter can be everted, e.g., by providing pressure to the inside of the rolling membrane and moving one end of the rolling membrane distally. This can cause an eversion of the rolling membrane along the vessel walls, for example, without substantive friction. The one end of the rolling membrane can be attached to, e.g., an inner shaft which moves distally when the rolling membrane, attached to a distal end of the inner shaft, is everted. This everts the rolling membrane in a distal direction towards and possibly past a stenosis. The as-inserted rolling membrane can for example widen the stenosis or provide a guide catheter for other components to be introduced to the blood vessel at the location of the stenosis.

However, there are a few issues that require care when using rolling membrane catheters.

First, the eversion of the rolling membrane is to be accompanied by a certain pressure applied to the rolling membrane. If a user, e.g., a health professional introducing the catheter, sets a pressure that is too high, the burst pressure of the rolling membrane can be exceeded, such that the rolling membrane might burst within the cardiovascular system of the patient. A frictionless retraction of the rolling membrane is not possible anymore and that can lead to an embolization of device components if the friction exceeds the tensile strength of the catheter backbone.

Second, if used with a guidewire, the rolling membrane, when in an inflated state, typically clamps the guidewire. Due to the everting motion of the rolling membrane, the guidewire advances twice as fast as the leading edge of the everting rolling membrane. This requires particular care to control the advancement of the guidewire such as to avoid the risk to perforate the cardiovascular system of the patient.

Third, a certain pressure inside the rolling membrane is required during retraction of the rolling membrane through the vessel. In case the rolling membrane is deflated or the pressure inside the rolling membrane is too low, the rolling membrane can be rendered unusable or damaged during retraction. Thus, particular care to control the retraction of the rolling membrane is required.

### SUMMARY OF THE INVENTION

A rolling membrane catheter includes a first rolling membrane. A motion unit is configured to determine a motion state of the first rolling membrane. The rolling membrane catheter can couple with a fluid unit for determining a fluid pressure and/or a fluid flow of the first rolling membrane. The rolling membrane catheter can be adapted to automatically couple the fluid unit and the motion unit for synchronized operation at least in part based on the motion state, the fluid pressure, and/or the fluid flow.

### BRIEF DESCRIPTION OF THE DRAWINGS

The Figures of the present disclosure show the following:

FIG. 1A Exemplary embodiment of a rolling membrane catheter including a motion unit, and a guidewire unit including a rolling membrane;

FIG. 1B Exemplary embodiment of a rolling membrane catheter including coupling to a fluid unit, a motion unit, a guidewire unit including a rolling membrane, a catheter balloon, and a balloon fluid unit;

FIG. 1C Overview of exemplary embodiment of a rolling membrane catheter including coupling to a fluid unit, a motion unit, a guidewire unit including a rolling membrane, a catheter balloon, and a balloon fluid unit with exemplary dimensions indicated;

FIG. 2 Exemplary scheme of chiseling motion of the guidewire of a rolling membrane catheter to open a narrow stenosis.

FIG. 3 Exemplary system including a rolling membrane catheter controlled by a control unit controlled by a patient via a user interface and operably coupled to an AI-assisted imaging evaluation.

### DETAILED DESCRIPTION OF THE PREFERRED EMBODIMENTS

According to a first aspect, a rolling membrane catheter with a first rolling membrane is provided. The rolling membrane catheter can further include a motion unit for determining a motion state of the first rolling membrane. The rolling membrane catheter can be adapted for coupling with a fluid unit for determining a fluid pressure and/or a fluid flow of the first rolling membrane.

As the volume to be inflated provided by the rolling membrane can increase during the eversion of the rolling membrane and reduce during the retraction of the rolling membrane, it can be advantageous to determine the fluid pressure and/or the fluid flow at least in part based on to the motion state and/or vice versa. For example, when a too high fluid pressure is provided, the rolling membrane can burst and when a too low pressure is provided, the rolling membrane can fold and/or be clinched and be everted or retracted in a non-ideal way. Therefore, it is beneficial to determine the motion state and/or the fluid pressure and/or the fluid flow of the first rolling membrane as it allows a safe operation of the rolling membrane catheter, for example an operation of the rolling membrane catheter at a non-critical motion state and/or fluid pressure and/or fluid flow at which the rolling membrane does not burst or clinch. Further, it can allow a user-friendly and flexible control of the rolling membrane catheter and a reduction of treatment time.

Generally, the term ‘fluid state’ as used herein can relate to the fluid pressure and/or fluid flow provided to a volume enclosed by the rolling membrane. A fluid can be a liquid and/or a gas. The term ‘motion state’ can relate to one or more parameters like a position, a velocity, an acceleration that characterizes the motion state of the rolling membrane (e.g. relative to (an outer shaft of) the catheter), or any mean values of these parameters over time. Further, the term ‘determine’ (a state or corresponding parameters) can relate to measuring and/or setting (a state or corresponding parameters).

In an example, the fluid unit can include one or more components configured to set a fluid state of the rolling membrane, e.g., by providing a constant or varying fluid pressure and/or a constant or varying fluid flow. In another example the fluid unit can include a sensor sensing parameters associated with a fluid state. The fluid unit can also include a plurality of subunits, e.g., at least one sensor sensing parameters associated with the fluid state and/or at least one component configured to set the fluid state, e.g., a pressure pump and/or a pressure reservoir configured to provide a constant or varying fluid pressure and/or a constant or varying fluid flow. The fluid unit can be coupled to the rolling membrane catheter by a tube, for example.

In an example, the motion unit can include a component configured to set a motion state, e.g., an actuator setting a certain position, inducing motion with a constant or varying velocity and/or constant or varying acceleration. Additionally or alternatively, the motion unit can include a sensor sensing one or more parameters associated with the motion state, like the position of the rolling membrane, e.g., by measuring the relative displacement between an inner shaft of the rolling membrane catheter and an outer shaft of the rolling membrane catheter, the velocity of the rolling membrane or other members of the rolling membrane catheter, or the acceleration of the rolling membrane or other members of the rolling membrane catheter. Generally, the motion unit can also include multiple subunits, such as at least one sensor sensing parameters like position, velocity, and/or acceleration and/or at least one actuator setting parameters like position, velocity, and/or acceleration and inducing the according motion of the rolling membrane.

In an example, the rolling membrane catheter and/or the motion unit can include a control unit, wherein the control unit is communicatively coupled to the motion unit or subunits of the motion unit (e.g., actuator(s) or sensor(s)) and the fluid unit or subunits of the fluid unit (e.g., pressure pump or sensor(s)).

In some examples, the rolling membrane catheter is adapted to output a warning signal based on the motion state and/or the fluid pressure and/or the fluid flow.

The fluid state can be sensed by a sensor of the fluid unit, for example, in response to a certain motion state determined by the motion unit. It can then provide feedback, e.g., to the operator to indicate whether the fluid state is within a range acceptable for the motion state (or vice versa). This can be done, e.g., via a display, an acoustic signal, etc. For example, a warning signal can be output when any value exceeds a predetermined threshold. Such threshold can be dependent on the motion state (fluid state).

The motion state can be sensed by a sensor of the motion unit, for example, in response to a certain fluid state determined by the fluid unit. It can then provide feedback, e.g., to the operator to indicate whether the motion state is within a range acceptable for the fluid state (or vice versa). This can be done, e.g., via a display, an acoustic signal, etc. For example, a warning signal can be output when any value exceeds a predetermined threshold. Such threshold can be dependent on the fluid state (motion state).

In one example, the rolling membrane catheter can include a lamp and/or a display for showing a visual warning signal and/or a component (e.g., a loudspeaker) for outputting an acoustic warning signal and/or a component for outputting vibrations which can be haptically sensed by the user as a warning signal.

In one example, the rolling membrane catheter and/or the motion unit can include a control unit, wherein the control unit is communicatively coupled to the motion unit or subunits of the motion unit (e.g., actuator(s) or sensor(s)) and the fluid unit or subunits of the fluid unit (e.g., pressure pump or sensor(s)). The control unit is configured to trigger a warning signal. The warning signal is then outputted by a component for outputting a warning signal (e.g., lamp, display, loudspeaker and/or a component for outputting vibrations which can be haptically sensed by the user as a warning signal). The control unit can be by wire or wirelessly communicatively coupled to the motion unit or subunits of the motion unit, the fluid unit or subunits of the fluid unit and/or the a component for outputting a warning signal.

Accordingly, a warning signal can be outputted once the rolling membrane catheter approaches a critical motion state and/or fluid pressure and/or fluid flow during operation.

Any such configuration can allow for user-friendly operation of the rolling membrane catheter as the motion state and/or fluid state is measured during operation and indicated on, e.g., a display or a warning signal is sent if a critical threshold can be approached. Generally, any such automatization can improve safety, for example by preventing the rolling membrane to burst or to clinch, allow user-friendly and flexible control of the rolling membrane catheter and can reduce the treatment time.

In some examples, the rolling membrane catheter is adapted such that as to automatically couple the fluid unit and the motion unit for synchronized operation at least in part based on the motion state, the fluid pressure, and/or the fluid flow.

As the desired fluid pressure and/or fluid flow of the rolling membrane can depend on the motion state of the rolling membrane, synchronizing operation of the motion and fluid units at least in part based on the motion state, the fluid pressure, and/or the fluid flow can strongly improve operation of the rolling membrane catheter. For example, the motion state of the rolling membrane can be automatically coupled to the fluid pressure provided to the rolling membrane. As the volume to be inflated provided by the rolling membrane can increase during the eversion of the rolling membrane and reduce during the retraction of the rolling membrane, it can be advantageous to determine the fluid pressure and/or the fluid flow at least in part based on to the motion state and/or vice versa. For example, when a too high fluid pressure is provided, the rolling membrane can burst and when a too low pressure is provided, the rolling membrane can fold and/or be clinched and be everted or retracted in a non-ideal way. Therefore, it can be beneficial to automatically couple the fluid unit and the motion unit for synchronized operation at least in part based on the motion state, the fluid pressure, and/or the fluid flow.

In an example, the fluid unit and the motion unit, that can each include one or more subunits, are coupled for synchronized operation. The synchronization of the fluid unit and the motion unit can rely on signaling between the two units and/or between subunits of the motion unit and/or the fluid unit. One or more individual subunits of each unit can further also be coupled for synchronous operation. In an example, the coupling can be achieved via a component configured to transmit signaling between the fluid unit and the motion unit or between any subunits thereof. The signal transmission can be directed either way. Also, simultaneous or alternating signaling in both directions is possible. The coupling can involve time delays due to signal propagation time and/or processing time, and synchronization of one (sub-)unit with any other (sub-)unit can therefore occur with an according time delay. The signaling can be direct and/or indirect, e.g., via a control unit (such as a microprocessor, a microcontroller, etc.) that can be part of the rolling membrane catheter (e.g., a subunit of the motion unit) or provided separately.

### By Way of Example, the Control Unit Can Be Implemented As a “processing System”

that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system can execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described can be implemented in hardware, software, or any combination thereof. If implemented in software, the functions can be stored on or encoded as one or more instructions or code on a computer-readable medium. Computer-readable media includes computer storage media. Storage media can be any available media that can be accessed by a computer. By way of example, and not limitation, such computer-readable media can include a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

The signals transmitted can at least partially be based on the parameter(s) associated with the fluid state and/or the motion state. The parameter(s) can be that set by an actuator and/or a component configured to set fluid pressure and/or fluid flow. Also, the parameter(s) can be that measured by a sensor sensing parameters associated with the motion state and/or the fluid state.

In some examples, the fluid unit can automatically adjust the fluid pressure provided to the rolling membrane and/or the fluid flow at least in part based on the motion state in such a way that the eversion and/or the retraction of the rolling membrane occurs at any time at a suitable inflation of the rolling membrane. This can occur simultaneously with sensing of the fluid state or without sensing it via a sensor.

In some examples, the rolling membrane catheter can be adapted to control the fluid unit based at least in part on the motion state to automatically determine the fluid pressure and/or fluid flow of the rolling membrane. This can be done by signaling provided by the rolling membrane catheter and/or the motion unit. The signaling is provided to the fluid unit directly, or it is provided indirectly by a control unit (of the catheter or external), optionally processed, and then provided to the fluid unit.

For example, the motion state of the rolling membrane can be determined by the motion unit as described herein. The determination of the motion state can, e.g., occur by setting the motion state via a component configured to set a motion state, sensed by a sensor, setting the motion state via a component configured to set a motion state and sensing the motion state by a sensor in a closed-loop configuration, and/or by a health professional or any user operating the rolling membrane. This can apply to both, the eversion of the rolling membrane and the retraction of the rolling membrane. In an example, signaling, at least in part based on the motion state, determined as described herein, and/or including information on parameters associated with the motion state, can be transmitted to the fluid unit. The signal transmission can occur in a mechanic, wireless, electronic and/or any other suitable way.

For example, one or more subunits of the fluid unit can be automatically adjusted to the motion state, automatically sense the fluid state via a sensor and/or automatically adjust the fluid state via a component configured to set a fluid state.

Generally, various configurations are possible in which signals are sent from an actuator inducing a motion state and/or a sensor sensing a motion state to the fluid unit including at least one sensor sensing parameters associated with the fluid state and/or a component for setting a fluid state. Any such configuration can allow for user-friendly operation of the rolling membrane catheter as the fluid state is either controlled automatically or measured during operation and potentially indicated on, e.g., a display or a warning signal is sent if a critical threshold can be approached. Generally, any such automatization can improve safety, for example by preventing the rolling membrane to burst or to clinch, allow user-friendly and flexible control of the rolling membrane catheter and can reduce the treatment time.

The rolling membrane catheter can, depending on user input via, e.g., a user interface, realize multiple of these operation modes. A user could for example decide whether he wants to enable automatic control of the fluid state, feedback via, e.g., a display on the current fluid state, or only to receive a warning signal should they approach a critical fluid pressure and/or fluid flow during operation. In an example, the fluid unit can be configured to control the fluid state such that at least one parameter associated with the fluid state remains within a given range. In this example, the user could, e.g., determine the fluid state himself and the fluid unit would only switch in an automatic control mode, should the user otherwise control the fluid unit such that at least one parameter associated with the fluid state would leave a given range in which safe operation can be ensured.

It is noted that the aspects herein can be used with a manual motion control (e.g. the motion unit merely includes one or more sensors) or with a (semi)-automatic motion control (e.g. the motion unit includes one or more actuators that can be motor-controlled, for example, e.g. based on corresponding commands by the user, e.g. via one or more buttons, a touchscreen, etc., that the catheter is provided with or that can be in operative connection with the catheter).

In some examples, the rolling membrane catheter can further include a component for controlling the motion unit based at least in part on the fluid pressure and/or fluid flow to automatically determine the motion state of the first rolling membrane. This can be done by signaling provided by the rolling membrane catheter and/or the fluid unit. The signaling is provided to the motion unit directly, or it is provided indirectly via a control unit (of the catheter or external), optionally processed, and then provided to the motion unit.

For example, the fluid state of the rolling membrane can be determined by the fluid unit as described herein. The determination of the fluid state can, e.g., occur by setting the fluid state via a component configured to set a fluid state, sensed by a sensor, setting the fluid state via a component configured to set a fluid state and sensing the fluid state by a sensor in a closed-loop configuration and/or by a health professional or any user operating the catheter. This can apply to both, the eversion of the rolling membrane and the retraction of the rolling membrane.

In an example, signaling, at least in part based on the fluid state, determined as described herein, and/or including information on parameters associated with the fluid state, can be transmitted to the motion unit. The signal transmission can occur in a mechanic, wireless, electronic and/or any other suitable way.

For example, one or more subunits of the motion unit can be automatically adjusted to the fluid state, e.g., automatically sense the motion state via a sensor and/or automatically adjust the motion state via a component configured to set a motion state. In one example, the motion state can be sensed by a sensor of the motion unit in response to a certain fluid state determined by the fluid unit. It can then provide feedback, e.g., to the operator to indicate whether the motion state is within a range acceptable for the fluid state (or vice versa). This can be done, e.g., via a display, an acoustic signal, etc. For example, a warning signal can be output when any value exceeds a predetermined threshold. Such threshold can be dependent on the fluid state (motion state). In some examples, the motion unit can automatically adjust the motion state of the rolling membrane in such a way that the eversion and/or the retraction of the rolling membrane occurs at any time at a suitable inflation of the rolling membrane. This can occur simultaneously with sensing of the motion state as described herein or without sensing it via a sensor.

Generally, any configuration is possible in which signals are sent from a component configured to set a fluid state and/or a sensor sensing a fluid state to the motion unit including at least one sensor sensing parameters associated with the motion state and/or a component for setting a motion state. Any such configuration can allow for user-friendly operation of the rolling membrane catheter as the motion state is either controlled automatically or measured during operation and potentially indicated on, e.g., a display or a warning signal is sent if a critical threshold can be approached. Generally, any such automatization can improve safety, for example by preventing the rolling membrane to burst or to clinch, allow user-friendly and flexible control of the rolling membrane catheter and can reduce the treatment time.

The rolling membrane catheter can, depending on user input via, e.g., a user interface, realize multiple of these operation modes. A user could for example decide whether they want to enable automatic control of the motion state, feedback via, e.g., a display on the current motion state, or only to receive a warning signal should they approach a critical motion state during operation. In an example, the motion unit can be configured to control the motion state such that at least one parameter associated with the motion state remains within a given range. In this example, the user could, e.g., set the motion state himself and the motion unit would only switch in an automatic control mode, should the user otherwise control the fluid unit such that at least one parameter associated with the fluid state would leave a given range in which safe operation can be ensured.

It is noted that the aspects herein can be used with a manual fluid control (e.g. the fluid unit merely includes one or more sensors) or with a (semi)-automatic fluid control (e.g. the fluid unit includes one or more components to provide a fluid state that can be motor-or pump-controlled, for example).

In some examples, the rolling membrane catheter can be adapted to control the fluid unit such as to automatically set the fluid pressure (and/or fluid flow) to lie within a first range when the motion state includes motion in a distal direction and/or to control the fluid unit such as to automatically set the fluid pressure (and/or fluid flow) to lie within a second range when the motion state includes motion in a proximal direction.

For example, the control is provided by a componenet for controlling the fluid unit, which is provided in the motion unit, or via a separate control unit (of the catheter or possibly also external to the catheter).

For example, this can specifically optimize the fluid state to the eversion of the rolling membrane, accompanied by a motion state involving motion in the distal direction, and/or the retraction of the rolling membrane, accompanied by a motion state involving motion in the proximal direction. Different fluid states, especially different pressures can be ideal for eversion and retraction, respectively.

Generally, there can be safety measures to prevent the fluid unit and/or the motion unit to exceed given threshold parameters, e.g., a maximum speed, a maximum acceleration, a maximum fluid pressure at which the rolling membrane might burst, a maximum fluid flow, etc., and/or to fall below given threshold parameters, e.g., a minimum speed, a minimum acceleration, a minimum fluid pressure, a minimum fluid flow, etc. There can be a set of predetermined threshold values stored on the fluid unit and/or motion unit, and/or a control unit (of the catheter or a separate control unit). Such safety measures can allow for user-friendly, error-resistant, and safe operation of the rolling membrane catheter.

In an example, this can be especially advantageous when the inner shaft of the rolling membrane catheter is retracted. In this case an at least in part automated control of the fluid unit can prevent unwanted clinching of the rolling membrane while at the same time it can prevent bursting of the rolling membrane when under too high pressure.

Further, a rolling membrane catheter assembly including the rolling membrane catheter and the fluid unit is provided.

According to a second aspect, a rolling membrane catheter can include a first rolling membrane, a guidewire adapted to be arranged within the first rolling membrane, and a guidewire unit configurable to clamp and/or retract the guidewire when the first rolling membrane is in an at least partly inflated state.

This can allow separate control of the guidewire position even when the rolling membrane is everted and thus in an at least partly inflated state (e.g. a fully inflated state). In some examples, this can be done by a clamp clamping the guidewire once or repeatedly, e.g., such that in total the distal ends of the rolling membrane and the guidewire advance by the same or a similar distance.

In an example, such guidewire unit can include an actuator to induce motion of the guidewire in the proximal direction and/or keep the guidewire at a given position. This can counteract any other force on the guidewire in the distal direction. Typically, the guidewire unit can provide higher forces than those acting in the distal direction (e.g. caused by the at least partly inflated rolling membrane that clamps the guidewire and moves distally if the guidewire unit were absent or switched off). This can be advantageous to prevent unwanted advancement of the guidewire which bears the risk of perforating the cardiovascular system of the patient. It can also be used to retract the guidewire before the eversion of the rolling membrane or during the eversion of the rolling membrane to increase safety during the eversion.

In an exemplary embodiment, the rolling membrane catheter includes a first rolling membrane which is adapted such that the guidewire, when arranged within the first rolling membrane, is clamped by the first rolling membrane when the first rolling membrane is in the at least partly inflated state.

In an example, the first rolling membrane of the rolling membrane catheter can be in an at least partly inflated state when fluid pressure is provided to the first rolling membrane. In its at least partly inflated state, e.g., with a pressure above a first threshold p_{clamp,1} provided to the first rolling membrane, the first rolling membrane can clamp the guide wire which advances centrally through the first rolling membrane. The clamping force can be high enough that during the eversion of the first rolling membrane, the guidewire is moved with the first rolling membrane. As the first rolling membrane performs an everting motion but the guidewire performs an axial translation, the distal tip of the guidewire advances in the distal direction at twice the speed of the leading edge of the first rolling membrane. It can be advantageous to counteract the advancing guidewire by a guidewire unit. The guidewire unit can at any time induce a translation of the guidewire in the proximal direction and/or keep the guidewire at a constant position. The retraction of the guidewire might either occur when the first rolling membrane is in an at least partly deflated state in which the clamping force of the first rolling membrane on the guidewire is at least partly reduced or it can occur when the first rolling membrane is in a (fully) inflated state, e.g. used during regular eversion, in which the clamping force of the first rolling membrane on the guide wire is high. The guidewire unit can provide a force exceeding the clamping force of the first rolling membrane on the guide wire to retract the guide wire or hold it at a constant position. That is, even when the first rolling membrane is in an at least partly inflated state of in a fully inflated state, the guidewire position can be controlled independently from the motion of the first rolling membrane by the guidewire unit.

The rolling membrane catheter can include a guidewire unit that includes a second rolling membrane configured to clamp the guidewire when the second rolling membrane is in an at least partly inflated state.

The second rolling membrane can be controlled by a second fluid unit configured to provide pressure to the second rolling membrane. The second rolling membrane is provided around the guidewire, e.g., at a proximal position of the catheter. When the second rolling membrane is in an at least partly inflated state and a pressure of above a second threshold p_{clamp,2} is provided to the second rolling membrane by the second fluid unit, it can clamp the guidewire. Providing fluid pressure to the second rolling membrane can inflate the rolling membrane such as to clamp the guidewire and thus fix the position of the clamped guidewire. In some examples, the second rolling membrane can be adapted to be everted in proximal direction, such as to not only clamp the guidewire but to retract it. The force applied by the guidewire unit can exceed the force exerted on the guidewire by the first rolling membrane. This can be achieved by providing a higher fluid pressure to the second membrane than to the first rolling membrane. Using a second rolling membrane to counteract the effect of the first rolling membrane clamping the guidewire yields various advantages. For example, similar or the same control componenet can be used to control the fluid unit determining the fluid state of the first rolling membrane and a similar fluid unit determining the fluid state of the second rolling membrane. Both membranes can be controlled in an analogous way.

It is noted that the first and second aspects can also be combined. For example, a rolling membrane catheter (assembly) with a first rolling membrane is provided. The rolling membrane catheter can further include a motion unit for determining a motion state of the first rolling membrane. The rolling membrane catheter can be adapted for coupling with a fluid unit for determining a fluid pressure and/or a fluid flow of the first rolling membrane. Further, the rolling membrane catheter can be adapted such as to automatically couple the fluid unit and the motion unit for synchronized operation at least in part based on the motion state, the fluid pressure, and/or the fluid flow. It can further include a guidewire adapted to be arranged within the first rolling membrane and a guidewire unit configurable to retract the guidewire when the first rolling membrane is in an at least partly inflated state. The rolling membrane catheter can include any of the further features described with respect to the first and second aspects herein.

When combining first and second aspects, a particularly safe and easy-to-use operation of the catheter can be provided. It can be ensured that fluid and motion state of the membrane are synchronized and at the same time, independent of the motion and pressure state of the membrane, the guidewire can be positioned in a predetermined fashion.

In some examples, the rolling membrane catheter can be configured such that as to automatically couple the guidewire unit to at least one of the fluid unit and the motion unit for synchronized operation.

The motion unit and/or fluid unit on the one hand, and the guidewire unit on the other hand, can for example operate in a (semi)-automated way such that the fluid and/or motion units and the guidewire unit are alternatingly and/or simultaneously engaged. This can yield an alternating advancement of the guidewire via clamping by the everting first rolling membrane and retraction of the guidewire via the guidewire unit. Also, it can yield an eversion of the first rolling membrane without movement of the guidewire. For example, the eversion of the rolling membrane can occur at a constant pressure or at a varying pressure above a given value to ensure that the rolling membrane is in a suitable inflated state at any time. The retraction can then, e.g. occur intermittently by applying a retraction force exceeding the clamping force of the first rolling membrane on the guidewire. In some examples, the eversion of the rolling membrane can be (repeatedly) interrupted when the guidewire unit retracts the guidewire to minimize the friction between the guidewire and the first rolling membrane counteracting the retraction force. This can yield a safe operation without unexpected advancement of the guidewire and can minimize the risk of perforating a vessel wall and increase the speed at which the catheter can be introduced in a safe and user-friendly way.

In a further example, the guidewire unit is configured to transmit a position, a velocity and/or an acceleration that characterizes a motion state of the guidewire to the control unit or motion unit, and the control unit or motion unit is configured to monitor the position, the velocity and/or the acceleration that characterizes a motion state of the guidewire. This can further yield a safe operation of the guidewire within the vessel. Further other exemplary embodiments are possible in which, e.g., the motion sensor indicates the motion state indicating to the user that the guidewire unit can or should be engaged to retract the guidewire. In such configuration, a warning signal can be sent when the guidewire has advanced by a predetermined threshold beyond the leading edge of the first rolling membrane. Further, examples are possible in which the fluid unit of the first rolling membrane is coupled to the guidewire unit for synchronized operation such that the pressure in the first rolling membrane is reduced when the guidewire unit retracts the guidewire. The synchronization of fluid unit, motion unit, and/or guidewire unit yields multiple advantageous technical effects: The guidewire can be controlled such that no unwanted advancement occurs, pressures can be held in an ideal range at any time and the advancement of the catheter can occur in a well-controlled way. All this increases the over-all safety of the treatment, can save time, and prevents typical user-errors.

According to a third aspect, a computer program is provided including instructions which, when the program is executed, causes a rolling membrane catheter as outlined herein (e.g. according to the first aspect) to automatically couple the fluid unit and the motion unit for synchronized operation at least in part based on the motion state, the fluid pressure, and/or the fluid flow.

Such a computer program can provide support to the user by taking over individual controls of the rolling membrane catheter automatically as outlined herein. This can increase safety during the treatment and reduce the time required for the treatment. According to a fourth aspect, a computer program can include instructions which, when the program is executed, causes a rolling membrane catheter as outlined herein (e.g. according to the second aspect) to control the guidewire unit, such that the guidewire remains within a predetermined range, when the first rolling membrane is everted.

In an example, the computer program can instruct the guidewire unit to repeatedly retract the guidewire whenever the guidewire is advanced by a predetermined length with the everting first rolling membrane by which it can be clamped. In a possible computer program, the frequency of the retraction or the advancement length after which the guidewire is retracted can be set by predetermined parameters. Alternatively, or in addition, it can at least in part be based on parameters determined by the fluid unit and/or the motion unit of the rolling membrane catheter. This program can assist the user advantageously by controlling all parameters that the user can wish to be controlled automatically when introducing the rolling membrane catheter into the cardiovascular system of a patient. For example, it can ensure that the first rolling membrane is everted with a constant average speed with intermittent phases of retraction of the guidewire to keep its position fixed relative to the distal-most tip of the rolling membrane. The program can be adapted to allow selection of such eversion mode, and/or other eversion modes such as: a mode in which the guidewire position is fixed with respect to the catheter (i.e. the rolling membrane everts but the guidewire remains stationary), or the guidewire moves at twice the speed of the rolling membrane.

It is noted that programs is provided that include the third and fourth aspect.

A computer program is provided that includes instructions which, when the program is executed, controls the motion unit, fluid unit and/or guidewire unit according to at least one control parameter such that the guidewire is moved alternatingly in proximal and distal direction, respectively, to remove a stenosis (e.g. by performing a chiseling motion). Removing a stenosis includes to open a path through a total occlusion of the vessel.

Such chiseling motion can be induced in various ways. All the following operations can at least partially automatically adjust parameters like frequency, amplitude, and/or relative position to the stenosis, the catheter, and/or potentially other information on the patient and/or the used catheter.

Firstly, a chiseling motion might be induced by alternatingly inflating and deflating the first rolling membrane via the fluid unit. This can lead to an alternating distal and proximal motion of the guidewire clamped by the first rolling membrane.

Secondly, a repeated retraction of the rolling membrane inner shaft against its hydraulic forward force of the rolling membrane followed by a controlled sudden reduction of this retraction force and/or a retraction followed by a sudden release of the guidewire by the guidewire unit can induce such chiseling motion. In addition, the fluid pressure provided to the first rolling membrane can be adjusted in an anticyclic way. In any case, due to the fluid pressure provided to the first rolling membrane, the first rolling membrane is under increasing tension when the guidewire is clamped by the first rolling membrane and retracted by the guidewire unit. This potential energy can then be transformed into kinetic energy when the guidewire is released by the proximal rolling membrane and quickly advances towards the stenosis.

Thirdly, in some examples, the motion unit can move the inner shaft in a proximal direction relative to the outer shaft to set the first rolling membrane under tension, which can e.g. be in an inflated state and clamp the guide wire. Thus, also the guidewire can move in the proximal direction. When the inner shaft is released or quickly moved in a distal direction, the guidewire quickly advances towards the stenosis. Repeating these steps can result in a chiseling motion of the guide wire.

The above three examples can also be combined. Generally, any combination of automated and/or non-automated motion of any componenet included in the rolling membrane catheter, as described herein, can be utilized to perform such chiseling motion of the guidewire.

The rolling membrane can also work in a blunt mode, where the guide wire is partly retracted, so that only the soft rolling membrane pushes repeatedly against the occlusion.

It is especially advantageous to at least partially automize such chiseling motion as it can significantly decrease the treatment time required to open a stenosis, reduce the likelihood of errors due to mistakes by the user and offers a simple way to adjust parameters associated with the chiseling motion for example at least partially to the position, shape, and texture of the stenosis.

Further, performing an automated chiseling motion by a rolling membrane catheter is especially advantageous as the first rolling membrane clamping the guide wire centers the guidewire with respect to the cross section of the blood vessel at the location of the stenosis and thus the guidewire can hit the stenosis in the center far away from the vessel walls which is why chiseling to open a stenosis using a rolling membrane catheter is particularly safe and the risk of perforating the vessel wall is low. Further, using the guidewire as a tool to open the stenosis does not require any additional componenet the rolling membrane catheter can be equipped with.

Also, by having a (semi-)automatic control, it is also possible to reach new parameter regimes that cannot be accessed by manual operation, e.g. a high frequency chiseling operation that can drastically reduce treatment time.

In an example, a computer program to induce a chiseling motion of the guidewire is provided, further including an interface for receiving image data of the stenosis and selecting the at least one control parameter at least in part based on the image data. Selecting at least one control parameter in an automated way can improve safety, the suitability of the at least one control parameter, and reduce the risk of erroneous image evaluation by the health professional operating the rolling membrane catheter. Such image data can be evaluated, e.g., by comparison with images of previously treated stenoses, to adjust treatment parameters associated with the chiseling motion, e.g., frequency, amplitude, relative position to the stenosis, and/or the used catheter, for example at least partially to the position, shape, and/or texture of the stenosis.

An exemplary computer program can further base the selecting of parameters at least in part on a (machine learning) model that has been trained with a plurality of training data sets, each training data set including image data (e.g. X-ray image data, angiography image data, intravascular ultrasound image data or optical coherence tomography) of a stenosis, at least one control parameter which has been used for removing the stenosis, and optionally a performance value. Analogously, an according computer program is provided to provide at least one parameter associated with any other action performed by the catheter.

The evaluation of the image data of a stenosis and the respective parameter selection can be based on training via machine learning, e.g., techniques like linear regression, logistic regression, decision tree, support vector machine (SVM) algorithm, naive Bayes algorithm, k-nearest neighbors' algorithm, K-means, random forest algorithm, dimensionality reduction algorithms, gradient boosting algorithm, and Adaptive Boosting algorithm. This can facilitate the choice of suitable treatment parameters without the need for time-consuming and potentially error-prone image evaluation procedures.

Another aspect is a computer-implemented method for training an artificial intelligence model for predicting at least one control parameter for treating a stenosis (e.g. as outlined herein). The method can include: Inputting a plurality of training data sets to the model to train the model, each training data set including image data of a stenosis, at least one control parameter which has been used for removing the stenosis, and optionally a performance value.

The model can be based on techniques such as linear regression, logistic regression, decision tree, support vector machine (SVM) algorithm, naive Bayes algorithm, KNN algorithm, K-means, random forest algorithm, dimensionality reduction algorithms, gradient boosting algorithm, and AdaBoosting algorithm.

Another aspect is a computer-implemented method of predicting at least one control parameter for treating a stenosis (e.g. as outlined herein). The method can include: Predicting the at least one control parameter based on image data of the stenosis using an artificial intelligence model that has been trained as outlined herein.

Another aspect can be training data for training an artificial intelligence model for predicting at least one control parameter for treating a stenosis. The training data can include a plurality of training data sets, each training data set including image data of a stenosis, at least one control parameter which has been used for removing the stenosis, and optionally a performance value. Another aspect relates to a use of such training data for training an artificial intelligence model for predicting at least one control parameter for treating a stenosis.

The computer programs as outlined herein can be executed by a corresponding catheter having a processing unit, e.g. a microcontroller, a microprocessor, etc. In other examples, an external processing unit can execute the program to which the catheter as outlined herein can be coupled, e.g. wirelessly and/or in a wired manner. Preferably, the catheter can only be fluidly coupled to the external processing unit. It is noted that all functions described herein can be implemented as a corresponding functionality (componenet) of the apparatus described herein, as a corresponding step of the methods outlined herein and/or as a corresponding instruction of the computer programs outlined herein. Even if described with reference to an apparatus, method and/or computer program, the aspects outlined herein can be applied to the respective other of an apparatus, method and/or computer program.

Possible embodiments of the present invention will be described in the following with reference to the above figures. For brevity, only a few embodiments can be described. The skilled person will recognize that the specific features described with reference to these embodiments can be modified and combined differently and that individual features can also be omitted if they are not essential. The general explanations in the sections above will also be valid for the following more detailed explanations.

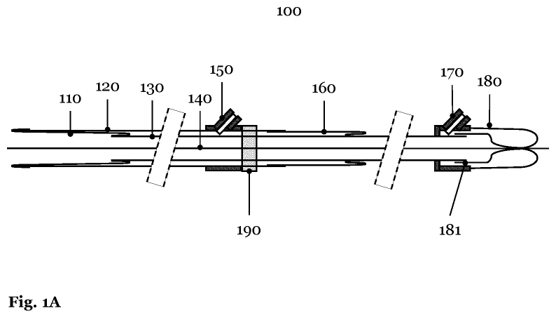

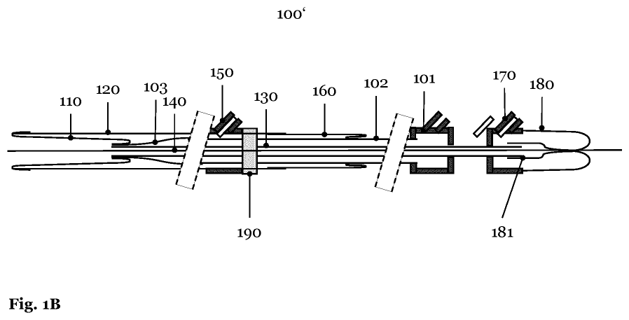

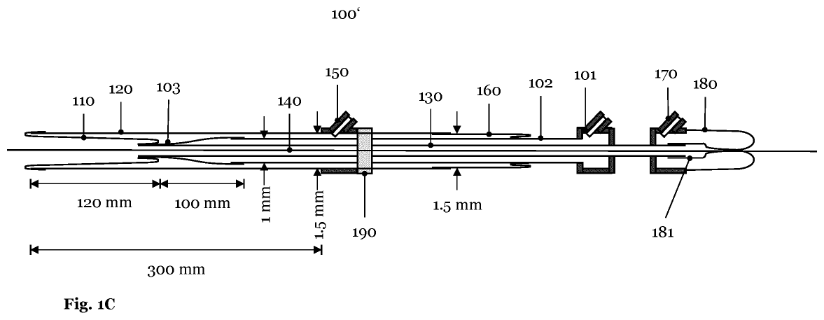

FIG. 1 A shows an exemplary embodiment of a rolling membrane catheter 100 including a componenet for coupling to a fluid unit 150, a motion unit 190, and a guidewire unit 170 including a second rolling membrane 180. It further includes an outer shaft 120 and an inner shaft 130 which is axially displaceable therein.

The first rolling membrane 110 of the rolling membrane catheter 100 is attached in a pressure-tight way to a distal end of the outer shaft 120 and to a distal end of the inner shaft 130. When the inner shaft 130 moves relative to the outer shaft 120 in the distal direction, the rolling membrane 110 can be everted, when appropriate pressure is provided to the first rolling membrane 110. In the exemplary embodiment, a merely optional third rolling membrane 160 is attached in a pressure-tight way to a proximal end of the outer shaft 120 and a proximal end of the inner shaft 130. The volume, the fluid pressure is provided to by the componenet for coupling 150 is defined by the inner shaft 130, the outer shaft 120, the first rolling membrane 110, and the third rolling membrane 160.

The motion unit 190 can include a sensor and/or a motion actuator that in this exemplary embodiment can measure and/or induce motion of the inner shaft 130 relative to the outer shaft 120, respectively. In some examples, the inner shaft 130 can be moved in distal and/or proximal direction manually and/or controlled by the motion unit 190.

The exemplary guidewire unit 170 of the rolling membrane catheter 100 shown in FIG. 1A is mounted to a proximal end region of the rolling membrane catheter 100. It includes a second rolling membrane 180 which can be attached to a separate axially movable shaft 181 that is still pressure tight towards the inner shaft 130-and configured to clamp the guide wire 140 when sufficient fluid pressure is provided to the second rolling membrane 180 which then is inflated and clamps the guidewire 140. When the second rolling membrane 180 is inflated, the second rolling membrane 180 can thus lock the guidewire 140. Additionally or alternatively, e.g. when the separate shaft 181 moves proximally, the second rolling membrane 180 can move the guidewire 140 proximally when the force acting onto the guidewire 140 via the second rolling membrane 180 exceeds the force acting onto the guidewire 140 in the distal direction via the first rolling membrane 110.

FIG. 1B shows another exemplary embodiment of a rolling membrane catheter 100′ including structure for coupling to a fluid unit 150, a motion unit 190, and a guidewire unit 170 including a second rolling membrane 180. It further includes an outer shaft 120 and an inner shaft 130 which is axially displaceable therein.

The first rolling membrane 110 of the rolling membrane catheter 100′ is attached in a pressure-tight way to a distal end of the outer shaft 120 and to a distal end of the inner shaft 130. When the inner shaft 130 moves relative to the outer shaft 120 in the distal direction, the rolling membrane 110 is everted. The exemplary rolling membrane catheter 100′ further includes a balloon 103 connected to the distal end of the inner shaft 130 and the distal end of an intermediate shaft 102. The intermediate shaft is further coupled to a balloon fluid unit 101 configured to provide fluid pressure to the balloon 103 to inflate the balloon 103. The balloon 103, in an inflated state, can widen stenoses. In the exemplary embodiment, a third rolling membrane 160 is attached in a pressure-tight way to a proximal end of the outer shaft 120 and a proximal end of the intermediate shaft 102. The volume, the fluid pressure is provided to is defined by the intermediate shaft 102, the outer shaft 120, the first rolling membrane 110, the balloon 103, and the third rolling membrane 160.

The motion unit 190 can include a sensor and/or a motion actuator that in this exemplary embodiment can measure and/or induce motion of the inner shaft 130 and the intermediate shaft 102 relative to the outer shaft 120, respectively. Said sensor of the motion unit 190 can measure a position, a velocity, an acceleration that characterizes the motion state of the rolling membrane 110 (e.g. relative to (an outer shaft 120 of) the catheter 100), or any mean values of these parameters over time.

The fluid 150 can include a fluid pressure sensor and/or a fluid flow sensor. The rolling membrane catheter 100 can include a lamp and/or a display for showing a visual warning signal and/or a loudspeaker for outputting an acoustic warning signal and/or a component for outputting vibrations which can be haptically sensed by the user as a warning signal. Said warning signal can be outputted if the motion state and/or fluid pressure and/or fluid flow reaches a threshold associated with a critical motion state, respectively, fluid pressure or fluid flow.

The exemplary guidewire unit 170 of the rolling membrane catheter 100′ shown in FIG. 1B is mounted to a proximal end region of the rolling membrane catheter 100. It includes a second rolling membrane 180 which can be attached to a separate axially movable shaft 181 and configured to clamp the guide wire 140 when sufficient fluid pressure is provided to the second rolling membrane 180 which then is inflated and clamps the guidewire 140. When the second rolling membrane 180 is everted, it can retract the guidewire 140 in the proximal direction. This can be the case when the force acting onto the guidewire 140 in the proximal direction via the second rolling membrane 180 exceeds the force acting onto the guidewire 140 in the distal direction via the first rolling membrane 110. Further, in the exemplary embodiment shown in FIG. 1B, the balloon fluid unit 101 is mounted to a proximal end region of the rolling membrane catheter 100′.

FIG. 1C provides a full overview of the exemplary embodiment of a rolling membrane catheter 100′ from FIG. 1B and typical dimensions are indicated: The outer shaft 120 can have a diameter of approximately 0.5 mm to 2.5 mm, for example 1.3 mm to 1.7 mm and the intermediate shaft 102 can have a diameter of approximately 0.3 mm to 1.7 mm, for example 0.8 mm to 1.2 mm. The length of the first rolling membrane 110 can be approximately 50 mm to 250 mm or about 100 to 140 mm and the balloon 103 can have an axial length of approximately 40 mm to 200 mm or about 80 to 120 mm. However, other embodiments can have different dimensions in general and/or with respect to individual components. For example, the length of the outer shaft 120 can be much longer than 300 mm, typically it can be up to 1300 mm.

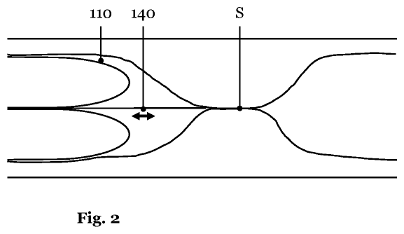

FIG. 2 shows an exemplary scheme of the guidewire 140 of a rolling membrane catheter 100 performing a chiseling motion to open a narrow stenosis S. When a stenosis S is too narrow for the rolling membrane 110 of a rolling membrane catheter 100 to be everted through it or even totally occluded, it can be necessary to open the stenosis S at least partially to create an opening through which the rolling membrane catheter 100 can be introduced, e.g., by everting the rolling membrane 110.

The guidewire 140 can be used as a tool to open such narrow stenoses S by causing it to perform a repeated, chiseling motion against the stenosis S. Parameters like frequency, amplitude, and/or relative position to the stenosis S can be adjusted based at least in part on position, shape, and texture of the stenosis S, the used catheter 100, and/or potentially other information on the patient and/or the used catheter.

It is emphasized that the aspects outlined hereinbefore with respect to an apparatus is provided as features or instructions of a computer program and/or as methods steps and vice versa.

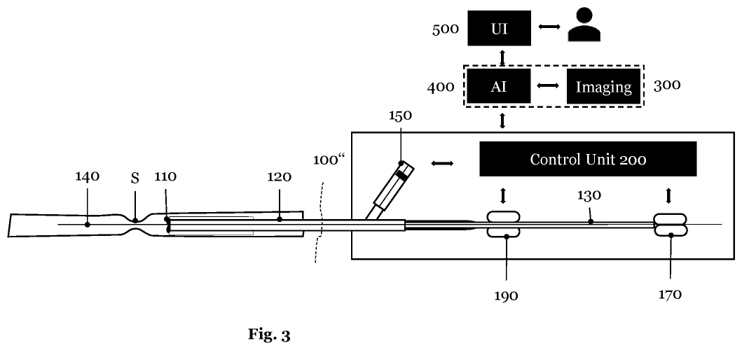

FIG. 3 shows an exemplary embodiment including an exemplary rolling membrane catheter 100″. The rolling membrane catheter 100″ includes a rolling membrane 110, a guidewire 140, an outer shaft 120 and an inner shaft 130. The motion unit 190 can determine the motion state of the inner shaft 130 and the guidewire unit 170 can determine the motion state of the guidewire 140. The guidewire unit 170 in the embodiment shown in FIG. 3 can determine the translation in the distal direction, the proximal direction and/or rotation of the guidewire 140. It can thus be seen as a further motion unit capable of more operations than simply to retract the guidewire 140. The rolling membrane catheter 100″ further includes structure for coupling to a fluid unit 150 configured as described herein.

In the exemplary embodiment shown in FIG. 3, the structure for coupling to the fluid unit 150, the fluid unit itself, the guidewire unit 170, and/or the motion unit 190 are operably coupled to a control unit 200 as described herein. The control unit 200 can cause the (structure for coupling to the) fluid unit 150, the motion unit 190, and/or the guidewire unit 170 to fulfil the functions described herein by determining the respective motion state and/or the respective fluid state. This can occur automatically, and/or the control unit 200 can be controlled by a user via a user interface 500.

In some embodiments, the control unit 200 can be configured to receive input via, e.g., the user interface 500 at least in part based on a mode that the user wants to perform. Exemplary modes include progression of the rolling membrane in the distal direction, compensation of the guidewire 140 advancement of twice the speed of the leading edge of the rolling membrane 110, retraction of the rolling membrane 110 without clinching, and/or the chiseling motion of the guidewire 140 for opening a stenosis S. It can further receive signaling based at least in part on the motion state and the fluid state of the rolling membrane 110, of the guidewire unit 170, and/or any other member of which the fluid and/or motion state is determined. The control unit 200 can then control any parameter associated with the fluid state and/or motion state via the respective units causing the catheter 100″ to perform the according mode, e.g., a chiseling motion of the guidewire 140 at a suitable frequency, position, and/or amplitude. In a simple embodiment, the exemplary modes are performed according to predetermined parameters associated with the respective modes.

In some examples, at least one parameter associated with at least one of the exemplary modes can be adjusted to a set of mode parameters, according to which the respective mode can be executed. This exemplary set of mode parameters can for example be provided by a health professional or be predetermined within at least one part of the system.

Additionally or alternatively, a set of input parameters can describe the treatment site, e.g., a stenosis S. The set of input parameters can relate to the location, size, texture, length, diameter, whether or not it is calcified and/or fibrotic, etc. of the stenosis S or any other treatment site. The exemplary set of input parameters can be based at least in part on imaging data, e.g. of the stenosis S, e.g. as described herein. For each set of input parameters, there can be an ideal set of mode parameters, which can be retrieved from a look-up table.

Additionally or alternatively, the control unit 200 can receive input from an optional AI-assistant 400 configured to evaluate imaging data 300 and thus provide a corresponding set of mode parameters. For example, the user can select, via the user interface 500 that a chiseling mode shall be performed to remove stenosis S. AI-assistant can then provide the corresponding set of mode parameters based on imaging data 300 of the stenosis S.

The imaging data can in some examples be based at least in part on (contrast agent-based) X-ray angiography, computed tomography, optical coherence tomography, intravascular ultrasound imaging, and/or any combination thereof of a stenosis to be treated, for example.

AI-assistant 400 can include a trained model that has been trained with a plurality of training data sets, each training data set including image data of a stenosis S, at least one control parameter which has been used for removing the stenosis S, and optionally a performance value.

A performance value can include to a binary value that indicates whether a treatment was successful, it can include a treatment duration, a measure of the size of the created opening, a measure for the harm caused to the vessel walls, etc.

A control parameter can be any of the set of mode parameters used for the treatment, for example, a frequency and/or an amplitude of a chiseling motion, and/or a distance from the stenosis S from which the chiseling motion was performed.

Based on the training data, the model can be trained to find optimal parameters for treating a stenosis with a chiseling mode based on imaging data of the stenosis.

When, in an exemplary application, a health professional can provide the AI-assistant 400 with an image of the treatment site. It can then provide predictions and/or suggestions, e.g., for suitable mode parameters of the chiseling motion for that specific treatment site. These mode parameters can be constant or varying parameters as described herein and/or related to the motion state and/or fluid state of any member of the catheter 100. The catheter 100 and/or the respective units can automatically execute the respective mode according to the mode parameters determined by the AI-assistant 400 for the stenosis S known to the algorithm from the image data it was provided with. Alternatively or in addition, the user can have the freedom to set the mode parameters themselves based at least in part on the suggested mode parameters. The AI-assistant 400 algorithm can, however, also be applied to other modes described herein, including the eversion of the rolling membrane 110, guidewire 140 movement, and/or the retraction of the rolling membrane 110, e.g. such as to position the catheter correctly relative to a stenosis S before treatment begins.

Generally, the AI-assistant 400 can be able to identify relevant input parameters and set them into context in a way a health professional might not be able to. Such AI-assistant 400 is provided to all modes described herein.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims

1. A rolling membrane catheter, comprising:
a first rolling membrane, and a motion unit configured to determine a motion state of the first rolling membrane;
wherein the rolling membrane catheter is adapted to couple with a fluid unit that can determine a fluid pressure and/or a fluid flow of the first rolling membrane.

2. The rolling membrane catheter of claim 1, wherein the rolling membrane catheter is configured to output a warning signal based on the motion state and/or the fluid pressure and/or the fluid flow.

3. The rolling membrane catheter of claim 1, wherein the rolling membrane catheter is configured to automatically couple the fluid unit and the motion unit to create operation at least in part based on the motion state, the fluid pressure, and/or the fluid flow.

4. The rolling membrane catheter of claim 1, comprising means for controlling the fluid unit based at least in part on the motion state to automatically determine the fluid pressure and/or fluid flow of the rolling membrane.

5. The rolling membrane catheter of claim 4, comprising means for controlling the motion unit at least in part on the fluid pressure and/or fluid flow to automatically determine the motion state.

6. The rolling membrane catheter claim 5, wherein the means for controlling the fluid unit is adapted to control the fluid unit to automatically set the fluid pressure to lie within a first range when the motion state comprises motion in a distal direction and/or wherein the means for controlling the fluid unit adapted to control the fluid unit to automatically set the fluid pressure to lie within a second range when the motion state comprises motion in a proximal direction.

7. A rolling membrane catheter assembly, comprising:
the rolling membrane catheter of claim 1; and
the fluid unit.

8. A rolling membrane catheter, comprising:
a first rolling membrane;
a guidewire configured to be arranged within the first rolling membrane; and
a guidewire unit configured to clamp and/or retract the guidewire when the first rolling membrane is in an at least partly inflated state.

9. The rolling membrane catheter of claim 8, wherein the first rolling membrane configured such that the guidewire, when arranged within the first rolling membrane, is clamped by the first rolling membrane when the first rolling membrane in the at least partly inflated state.

10. The rolling membrane catheter of claim 1, comprising a guidewire configured to be arranged within the first rolling membrane; and
a guidewire unit configured to clamp and/or retract the guidewire when the first rolling membrane is in an at least partly inflated state, wherein the guidewire unit comprises a second rolling membrane configured to clamp the guidewire when the second rolling membrane is in an at least partly inflated state.

11. (canceled)

12. The rolling membrane catheter of claim 1, comprising a guidewire configured to be arranged within the first rolling membrane; and
a guidewire unit configured to clamp and/or retract the guidewire when the first rolling membrane is in an at least partly inflated state, wherein the rolling membrane catheter is configured to automatically couple the guidewire unit to at least one of the fluid unit and the motion unit to synchronize operation.

13. A computer program comprising instructions which, when the program is executed, causes the rolling membrane catheter of claim 7 to automatically couple the fluid unit and the motion unit to synchronize operation at least in part based on the motion state, the fluid pressure, and/or the fluid flow.

14. A computer program comprising instructions which, when the program is executed, causes the rolling membrane catheter of claim 12 to control the guidewire unit, such that the guidewire remains within a predetermined range, when the first rolling membrane is everted.

15. A computer program comprising instructions which, when the program is executed, causes the rolling membrane catheter of claim 12 to control the motion unit, fluid unit and/or guidewire unit according to at least one control parameter such that the guidewire is moved alternatingly in a proximal and distal direction, respectively, for removing a stenosis (S)

16. The computer program of claim 15, comprising an interface for receiving image data of the stenosis(S) and selecting the at least one control parameter at least in part based on the image data.

17. The computer program of claim 16, wherein the selecting is at least in part based on a model that has been trained with a plurality of training data sets, each training data set comprising image data of a stenosis, and at least one control parameter which has been used for removing the stenosis(S).
